(19) United States
(12) Patent Application Publication
(10) Pub. No.: US 2021/0060274 A1
(43) Pub. Date: Mar. 4, 2021

(54) AUTOMATED BAG-VALVE-MASK (BVM)

(71) Applicant: Keysight Technologies, Inc., Santa Rosa, CA (US)

(72) Inventors: Wai Seong Lim, Bayan Lepas (MY); Suang Chian Chai, Teluk Kumbar (MY); Keng Keat Yeoh, Prai (MY); Chee Yang Liew, Bukit Mertajam (MY)

(21) Appl. No.: 17/012,583

(22) Filed: Sep. 4, 2020

(51) Int. Cl. A61M 16/00

(52) U.S. Cl. CPC A61M 16/0078; A61M 2205/33; A61M 16/022

(57) ABSTRACT

To supplement the global shortage of mechanical ventilators caused by the Covid-19 pandemic, a cost-effective automated bag-valve-mask (BVM) device capable of producing regular repeatable ventilation to assist patient breathing is provided. The operation of compressing and decompressing a manual BVM device is automated using an automated compression fixture controlled by a digital programmable electronic control unit. The BVM airbag mounted to the automated compression fixture is placed between two finger levers that can perform cyclical compression and decompression to the BVM airbag, which is realized through a motor-driven gear mechanism. The automated BVM device allows different settings of ventilation parameters and is also associated with necessary safety measures. The automated BVM device may be built based on commercially available authority approved manual BVM devices such as the Ambu® resuscitators to reduce development cost. The simplified design and market availability of the components also enable its rapid manufacture in large quantities.

### BACKGROUND

Manual resuscitators, known as bag-valve-masks (BVMs) are widely used in medical and emergency treatments to assist patients who are unable to properly breathe. BVMs are designed to be handheld and manually compressed by a human operator at a predetermined rate to supply air to the patient. Typical use applications of the BVM are for maintaining patient respiration during emergency treatment or transportation. Since the worldwide outbreak of COVID-19 pandemic, the unprecedented large number of patients admitted to hospitals and intensive care units (ICUs) has resulted in a global shortage of mechanical ventilators. As a backup solution to mechanical ventilators, which are sophisticated, difficult to manufacture, and expensive, the less expensive and more available BVMs, when equipped with HEPA (High-Efficiency Particulate Air) filters, have been used to assist ventilation of patients with less critical conditions, or to assist in circumstances where the mechanical ventilators are not available. Relevant background references to BVMs include U.S. Pat. Nos. 5,222,491A, 8,534,282 B2, published patent applications US20050284472A1, WO2019229776A1, WO2019224810A1 and the non-patent reference David L. Chandler, “MIT team races to fill Covid-19-related ventilator shortage”, MIT News, Apr. 20, 2020, URL: http://news.mit.edu/2020/e-vent-covid-19-ventilator-shortage-0420.

When operating the BVM device, a medical provider typically holds the BVM with both hands and compresses the airbag portion of the BVM at a steady rate to provide ventilation to the patient at regular time intervals. Providing this type of ventilation assistance to a patient for a prolonged period of time is often not feasible because this manual compression may occupy critical medical personnel, and the regular prolonged manual compression of the airbag may be fatiguing. In addition, insufficient airbag compression may cause hypoventilation, while over-compression of the airbag may cause hyperventilation or gastric insufflation. To overcome the disadvantages of manual compression of BVMs, various systems have been developed to automate the operation of BVMs. For example, U.S. Pat. No. 5,222,491 discloses a modified BVM with guy wires attached inside the airbag that are tensioned cyclically by mechanical attachments and actuators that cause the airbag to expand and collapse. Published U.S. Patent Appl. US20050284472A1 and PCT Pat. Appl. WO2019229776A1 disclose other examples of BVM systems that are mounted in a housing and pressurized by either a piston-like member or motor-driven plate. U.S. Pat. No. 8,534,282 discloses an automated BVM system having the airbag is confined by straps and compressed by a member concavely shaped to correspond to at least a portion of convexly curved outer surface of the airbag. Published PCT Pat. Appl. WO2019224810A1 discloses a portable automated BVM system with the airbag compressed by means of a strap driven by a gear train.

Apart from the compression mechanism, it is crucial for an automated BVM system to provide precise control of the compression frequency and duration, as well as the air volume supplied by the BVM during each compression to avoid inconsistencies. It is also desirable to have flexibility to adjust ventilation parameters to accommodate the needs of different patients. Safety measures in response to emergency and device failure are also essential to avoid harm to patients. Further, the component availability and manufacture cost are import to the manufacturability of a BVM system, especially when there is a large demand in a very short time, such as during the COVID-19 pandemic situation.

In view of all the above-mentioned considerations, there is a need to provide a cost-effective automated BVM system that is capable of producing regular repeatable ventilation assistance to patients.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 shows an isometric view of an automated BVM system according to a representative embodiment of the present invention;

FIG. 2A shows an isometric view of the automated compression fixture 200 of an automated BVM system 10 according to embodiments of the present invention;

FIG. 2B shows a disassembled view of the automated compression fixture 200 of an BVM system according to embodiments of the present invention;

FIG. 3A shows an isometric view of mechanical construction of the motor-lever assembly 2100 in the automated compression fixture 200 according to embodiments of the present invention;

FIG. 3B shows a disassembled view of the motor-lever assembly 2100 according to embodiments of the present invention;

FIG. 4A is a schematic diagram of an illustrative operation scenario according to embodiments of the present invention, with the motor-lever assembly compressing the BVM to a compressed state;

FIG. 4B is a schematic diagram of an illustrative operation scenario according to embodiments of the present invention, with the motor-lever assembly decompressing the BVM to an original state;

FIG. 5 shows an isometric view of the electronic control unit of the BVM system according to embodiments of the present invention;

FIG. 6 is a schematic diagram showing the touch panel human-machine interface (HMI) of the electronic control unit according to embodiments of the present invention; and

FIG. 7 is a schematic block diagram of the electronic control unit according to embodiments of the present invention.

### DETAILED DESCRIPTION

A BVM system 10 constructed according to embodiments of the present invention includes equipping commercially available manual BVMs with an automated compression fixture 200 and an electronic control unit 300. The BVM system 10 provides for different settings of Tidal Volume (VT), Respiratory Rate (BPM), and Inspiratory/Expiration Time (I/E) Ratio and may also include associated safety measures such as alarms and an emergency stop mechanism. The BVM system 10 is suitable to meet the minimum clinical functionality to provide assisted ventilation to patients with less critical conditions and supplement the shortage of mechanical ventilators in the face of the Covid-19 pandemic. The design of the BVM system 10 and availability of components, enables rapid high-volume manufacturing of the BVM systems.

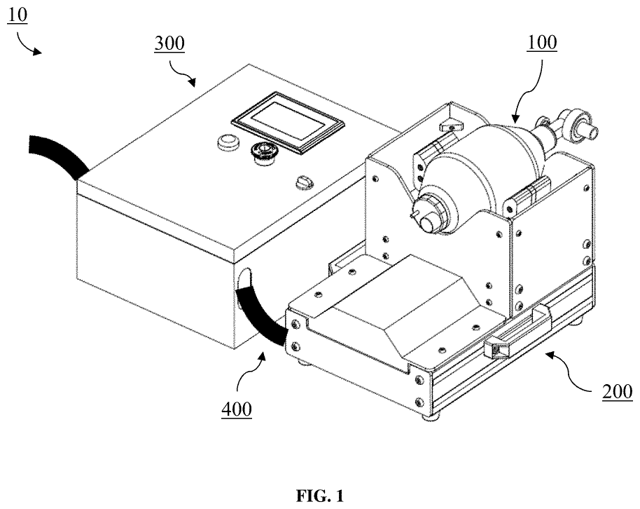

FIG. 1 shows a BVM system 10 comprising a compressible BVM 100, an automated compression fixture 200, and an electronic control unit 300. For the ease of manufacture, the compressible BVM 100 may be a commercially available BVM device such as an Ambu® resuscitator that is approved medical application. The automated compression fixture 200 provides a receiving structure to seat and apply regular repeatable compression to the compressible BVM 100. An electronic control unit 300 is coupled to the automated compression fixture 200 via cable connectors 400. The electronic control unit 300 is responsible for receiving user settings, supplying power to the automated compression fixture 200, as well as controlling the operation of the automated compression fixture 200.

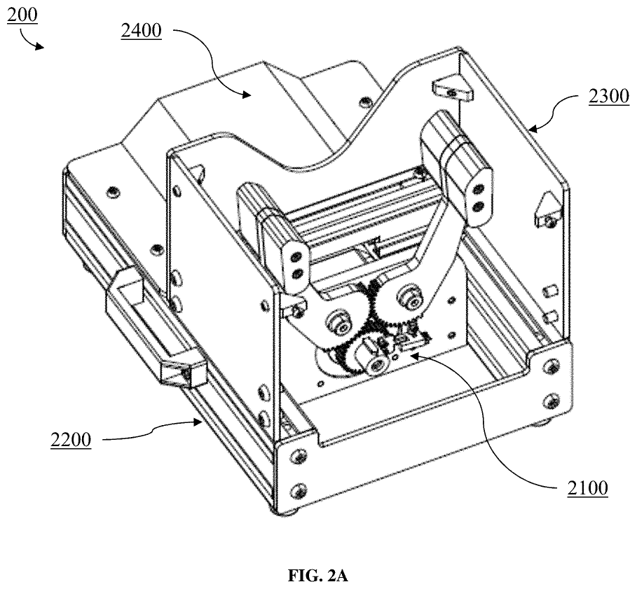

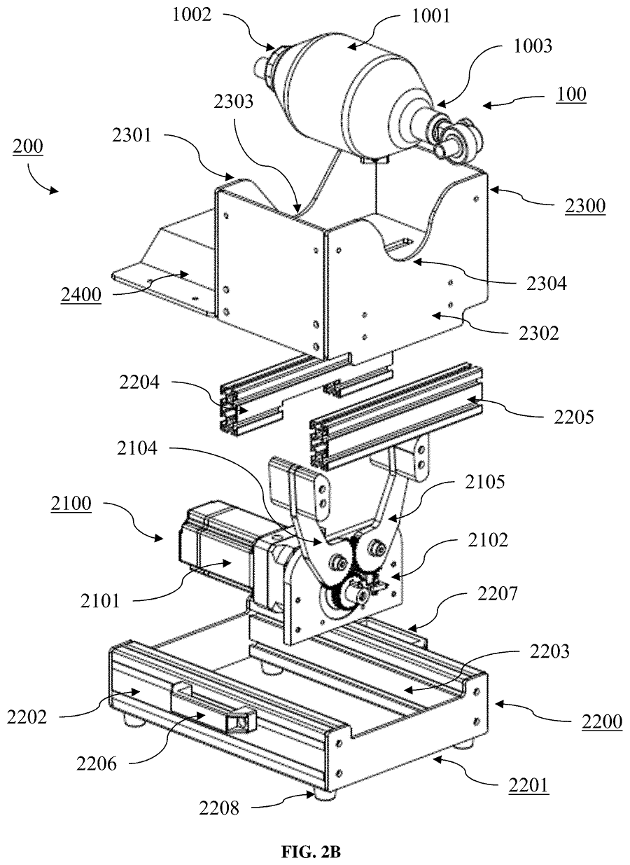

FIG. 2A shows an isometric view of the arrangement inside the automated compression fixture 200 shown in FIG. 1, with one side of the receiving structure 2300 removed. The automated compression fixture 200 comprises a motor-lever assembly 2100, a support base 2200, a receiving structure 2300 attached to the support base 2200, and a protective cover 2400 covering the motor (not shown).

FIG. 2B shows a more detailed view of the arrangement of components in the automated compression fixture 200, wherein the components disassembled. The support base 2200 comprises a rectangular frame 2201 that defines a space for receiving the motor-lever assembly 2100 inside the rectangular frame 2201. A first support member 2202 and a second support member 2203 form two parallel sides of the rectangular frame 2201. Preferably, the first and second support members 2202, 2203, which are perpendicular to the vertical mounting plate 2102 of the motor-lever assembly 2100, have a hollow structure to reduce the overall weight of the support base 2200 and to provide for easy attachment of the vertical mounting plate 2102 to the first and second support members 2202, 2203. Handles 2206, 2207 may be attached to the respective outward-facing vertical sides of the first and second support members 2202, 2203 to facilitate carrying the device. Protective bumpers 2208 may be attached to the four bottom corners of the rectangular frame 2201 to lift the device off the ground and buffer the vibrations during operation of the BVM system 10.

A motor-lever assembly 2100 is accommodated within the space defined by the support base 2200, by connecting the vertical mounting plate 2102 to the first and second support members 2202, 2203. A third support member 2204 and a fourth support member 2205 are parallel to each other and perpendicular to the first and second support members 2202, 2203. The support members 2204, 2205 are attached at their respective first and second distal ends to the upward-facing surface of the first and second support members 2202, 2203 to further strengthen the support base 2200 and to provide vertical attachment surfaces for the receiving structure 2300. First and second finger levers 2104, 2105 are pivotally attached to the front of the vertical mounting plate 2102. The third support member 2204 is placed behind the vertical mounting plate 2102 and over the motor 2101, whereas the fourth support member 2205 is placed in front of the motor-lever assembly 2100, with sufficient clearance to prevent interfering contact with any parts of the motor-lever assembly 2100. To avoid contact with any parts of the motor-lever assembly 2100, a portion of the lower part of the third support member 2204 may include a cut out to accommodate a portion of the motor. The space defined by the third and fourth support members 2204, 2205 ensures that the first and second finger levers 2104, 2105 can be operated without obstruction, and defines the distance between the outer vertical surface of the third and fourth support members 2204, 2205 to be approximately the distance between the inlet 1002 and outlet 1003 ends of the airbag 1001 of the BVM 100.

As shown in FIG. 2B, the receiving structure 2300 for mounting the airbag 1001 of the BVM 100 above and between the first and second finger levers 2104, 2105 may be a rectangular structure having a first side plate 2301 and a second side plate 2302 that are both parallel to the vertical mounting plate 2102. The respective upper portion of the first and second side plates 2301, 2302 have curves 2303, 2304 to seat either of the inlet and outlet ends 1002, 1003 of the airbag 1001 at the bottom of the curve. The radius of the curve on the first and second side plates 2301, 2302 may not be identical and may be larger than the radius of the respective ends 1002, 1003 of the airbag 1001. While no additional securing features are shown in the exemplary embodiment to secure the airbag 1001 of the BVM 100 to the receiving structure 2300, ordinary securing measures such as hooks or rubber straps may be used to further secure the airbag 1001 of the BVM to the receiving structure 2300. The protective cover 2400 behind the receiving structure 2300 is attached to the support base 2200 to cover the motor 2101 with sufficient mechanical clearance. The cover 2400 shields the motor 2101 from the external environment and may protect an operator of the BVM system 10 from the heat generated by the motor during operation.

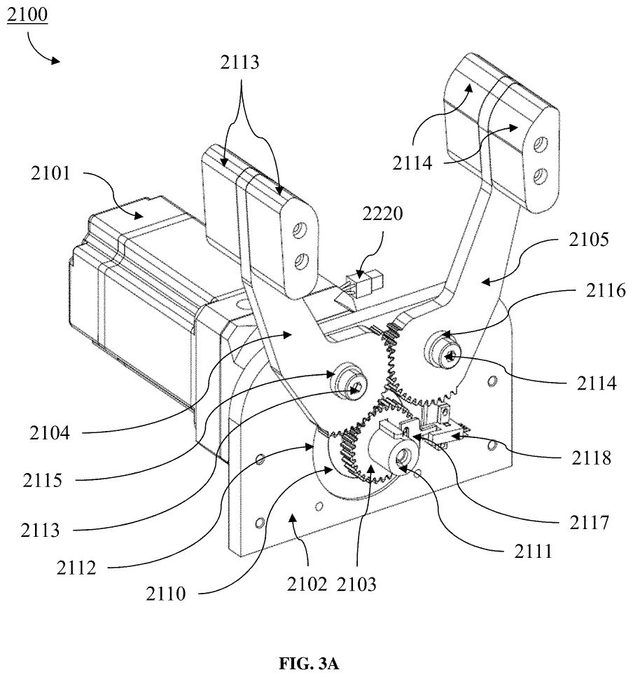

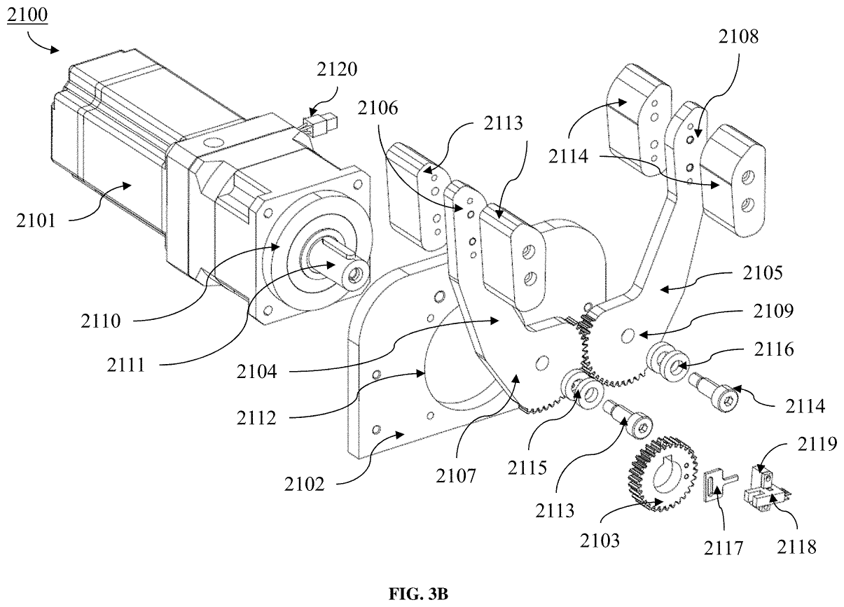

The motor-lever assembly (2100) is the core of the automated compression fixture 200 and enables cyclical compression and decompression of the airbag 1001 of the BVM 100. FIGS. 3A and 3B illustrate the detailed constructive arrangement of motor-lever assembly 2100 in an assembled and disassembled view, respectively. The first finger lever 2104 and the second finger lever 2105 are identical and customized to have an overall shape of a bending finger so that one side of each of the finger levers 2104, 2105 has a concave profile. Each of the finger levers 2104, 2105 has a distal end 2106, 2108 with a finger-pad profile at the concave side, and a proximal end 2107, 2109 with a gear profile. The two finger levers 2104, 2105 are positioned at the same height above the circular opening 2112 in the vertical mounting plate 2102 and are symmetric about the vertical central axis of vertical mounting plate 2102 with the concaved profiles facing each other. The finger-pad profiles at distal ends 2106, 2108 point upward, and the gear profiles at the proximal ends 2107, 2109 mesh with each other. The pivotal attachment of the finger levers 2104, 2105 to the front side of the vertical mounting plate 2102 is achieved with bolts 2113, 2114 passing through holes at the center of their gear profiles. Washers 2115, 2116 are used at both sides of the holes to minimize the friction between surfaces and the resistance during pivotal motion of the levers about the bolts 2113, 2114. Gripping elements 2113, 2114 are attached to each of the finger-pad profiles of the distal ends 2106, 2108 of the corresponding finger levers 2104, 2105 to increase the contact area with the airbag 1001. The shape and size of the finger levers 2104, 2105 is designated to ensure that when the finger levers are at a relaxed (IDLE) state, the finger-pad area of the distal ends 2106, 2108 should be at the level slightly above the horizontal central axis of the airbag 1001 of the BVM 100, and the distance between the proximal ends 2106, 2108 is slightly larger than the diameter of the airbag 1001.

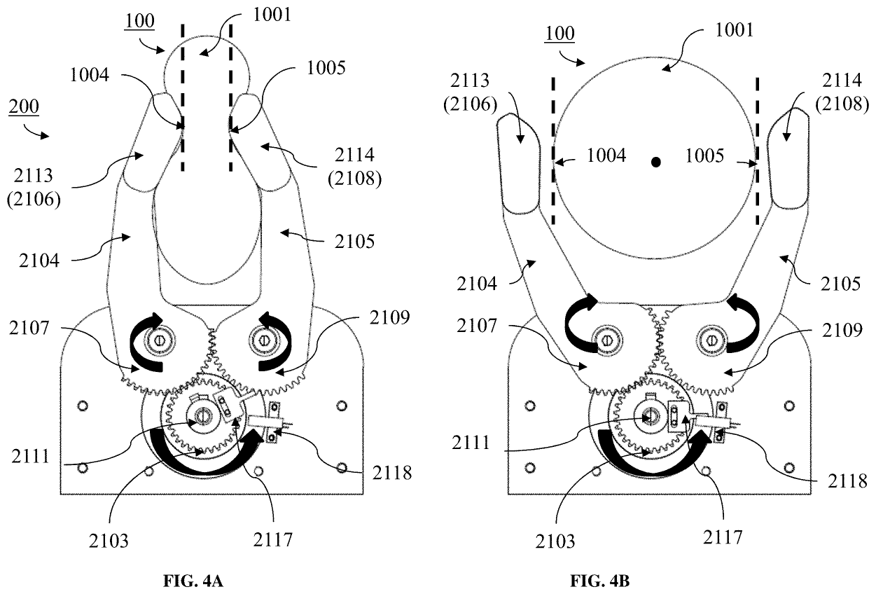

The motor 2101 has a circular mounting guide 2110 protruding from its mounting surface, and a driving shaft 2111 of the motor passes through the center of the mounting guide 2110. The motor 2101 is attached to the back side of the vertical mounting plate 2102 with the driving shaft 2111 passing through the circular opening 2112 in the vertical mounting plate 2102. A drive gear 2103 is installed on the driving shaft 2111 and locked by a projection on the driving shaft. The circular opening 2112 in the vertical mounting plate 2102 is slightly bigger than the circular mounting base 2110 on the motor 2101 so that the circular mounting base 2110 can be seated in the circular opening so that there is no obstruction to the rotation of the drive gear 2103. As illustrated by the example in FIG. 3A and FIG. 4A, the center of the driving shaft 2111, which is also the center of the drive gear 2103, is off the vertical central axis of the vertical mounting plate 2102 to its left side, making the drive gear 2103 mesh only with the gear-like proximal end 2107 of the first finger lever 2104. A gear train is therefore formed from the drive gear 2103 to the gear-like proximal end 2107 of the first finger lever 2104 followed by the gear-like proximal end 2109 of the second finger lever 2105. Similarly, the center of the drive gear 2103 may also be arranged to the right side of the vertical central axis of the vertical mounting plate 2102 so that the drive gear 2103 meshes only with the gear-like proximal end 2109 of the second finger lever 2105, causing a gear train from the drive gear 2103 to the gear-like proximal end 2109 of the second finger lever 2105 followed by to the gear-like proximal end 2107 of the first finger lever 2104. A photoelectric limit sensor 2118 mounted on a spacer 2119 is installed at the edge of the circular opening 2112 beside the drive gear 2103. A sensor flag 2117 with a projection from one side is attached to the front surface of drive gear 2103, positioned so that when the first and second finger levers 2104, 2105 open to predetermined maximum extent (e.g. at the original start position), the projection from the sensor flag 2117 is inserted in the U-shape slot of the limit sensor 2118. The limit sensor 2118 is used to protect the finger levers from over-opening beyond the defined maximum extent.

The materials selected for the support base 2200, the support members 2202, 2203, 2204, 2205, and the motor-lever assembly 2100 should be of good durability, and preferably, of light weight to reduce the overall weight of the device. Prior to real manufacture, the design of the automated compression fixture 200, the drive gear 2103, the first and second finger levers 2104, 2105 and the arrangement of parts forming the gear train should be simulated and tested with software such as CAD to ensure the design and arrangement is functional with the BVM 100 that is included in the BVM system 10.

FIGS. 4A and 4B illustrate exemplary operation scenarios with the automated compression fixture 200 compressing the airbag 1001 of BVM 100, and decompressing the airbag 1001 of the BVM 100, respectively, according to embodiments of the present invention. The movement of the first and second finger levers 2104, 2105 is done through the motor-driven gear mechanism. Referring to FIG. 4A, during the compression movement, the drive gear 2103 is rotated in the counterclockwise direction by the driving shaft 2111 of the motor 2101, and the projection from the sensor flag 2117 moves out from the U-shape slot of the limit sensor 2118. The drive gear 2103 transfers the rotational motion to the meshing gear-like proximal end 2107 of the first finger lever 2104 to make it rotate in the counterclockwise direction, which results in a moving motion of the finger-pad like distal end 2106 of the first finger lever 2104 and the attached gripping elements 2113 towards the airbag 1001 to make contact at a first outer surface 1004 of the airbag 1001 of the BVM 100. The rotating gear-like proximal end 2107 of the first finger lever 2104 further transfers its counterclockwise rotational motion to the meshing gear-like proximal end 2109 of the second finger lever 2105 and makes it rotate in the clockwise direction, resulting in a moving motion of the finger-pad like distal end 2108 of the first finger lever 2105 and the attached gripping elements 2114 towards the airbag 1001 to make contact at a second outer surface 1005 of the BVM airbag 1001. The simultaneous moving motion of the finger-pad like distal ends 2106, 2108 of the first and second finger levers 2104, 2105 result in a compression effect to the airbag 1001. The finger-pad like distal ends 2106, 2108 of the first and second finger levers 2104, 2105 keep moving towards each other until a predetermine compression extent is reached.

Referring to FIG. 4B, during the decompression movement, the drive gear 2103 is rotated in the clockwise direction by the driving shaft 2111 of the motor 2101. The drive gear 2103 transmits the rotational motion to the meshing gear-like proximal end of the first finger lever 2104 to make it rotate in the clockwise direction, which results in a moving motion of the finger-pad like distal end 2106 of the first finger lever 2104 and the attached gripping elements 2113 away from the airbag 1001. The rotating gear-like proximal end 2107 of the first finger lever 2104 further transmits its clockwise rotational motion to the meshing gear-like proximal end 2109 of the second finger lever 2105 and makes it rotate in the counterclockwise direction, resulting in a moving motion of the finger-pad like distal end 2108 of the first finger lever 2105 and the attached gripping elements 2114 away from the airbag 1001. The simultaneous moving motions of the finger-pad like distal ends 2106, 2108 of the first and second finger levers 2104, 2105 result in a decompression effect to the airbag 1001. The finger-pad like distal ends 2106, 2108 of the first and second finger levers 2104, 2105 keep moving away from each other until the first and second finger levers 2104, 2105 goes back to their original start position, wherein the projection from the sensor flag 2117 moves back into the U-shape slot of the limit sensor 2118 to trigger the stop of the movement and avoid any over-turning of the first and second finger levers 2104, 2105.

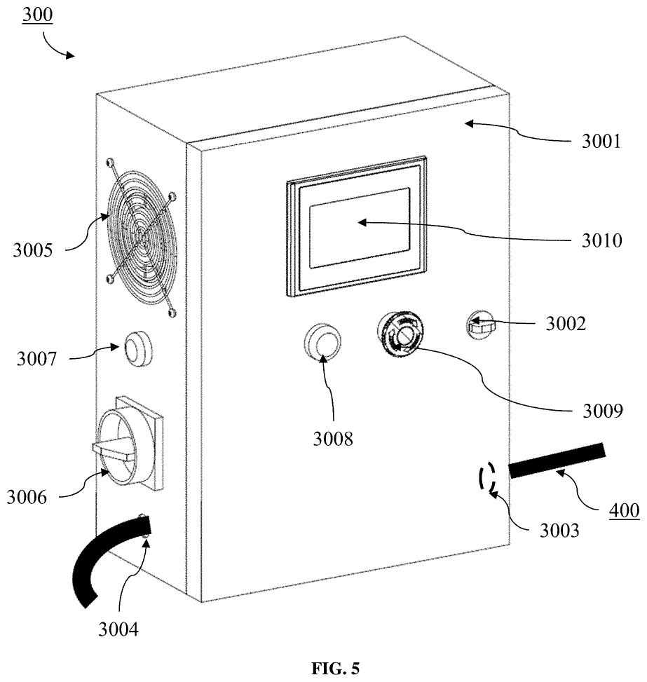

The operation of the automated compression fixture 200 is controlled by an electronic control unit 300 via cable connectors 400. Referring to the exemplary illustration as shown in FIG. 5, the electronic control unit comprises a control box 3001 with a lock 3002 that accommodates the electro-mechanical components used for power supply and operation control. A power switch 3006 is located at one side of the control box 3001 together with a pilot light indicator 3007 beside it, which would be lighted up when the device is powered up. A heat dissipation window 3005 located at the side of the control box 3001 is used to dissipate heat generated from the inside of the control box 3001. The cable connectors 400 for powering and controlling the automated compression fixture 200 extends out through a first cable hole 3003 located at one side of the control box 3001, while the power cable of the power supply unit 3020 inside the control box 3001 extends out of the control box 3001 through a second cable hole 3004 located at another side of the control box 3001. On the front door of the control box 3001, there is a buzzer 3008 for alerting system failure. An emergency stop button 3009 is located beside the buzzer 3008, which may be pushed to stop the operation of the motor-lever assembly 2100 in case of emergency or device failure. Once being pushed, the emergency stop button 3009 may be deactivated by simultaneously pushing and turning the button clockwise. There is also a touch panel human-machine interface (HMI) 3010 located at the front door of the control box 3001, which is used for displaying system information and receiving from a user the operational commands and ventilation operational parameter settings to control the operation of the automated compression fixture 200.

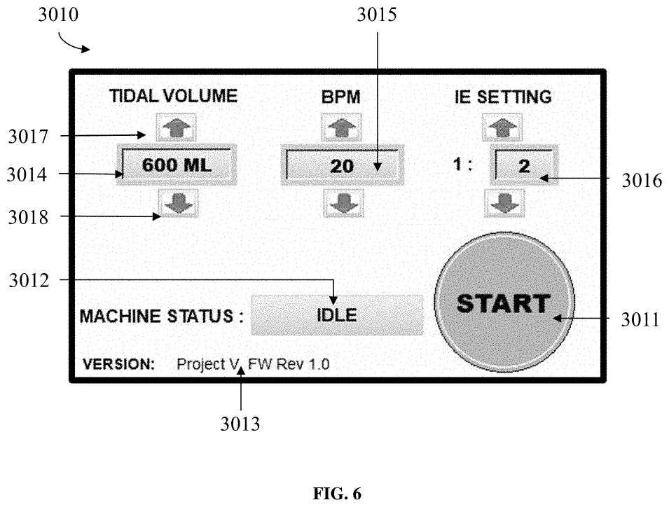

FIG. 6 shows the display of the touch panel HMI 3010 of the electronic control unit 300 according to a representative embodiment as shown in FIG. 5. The START/STOP button 3011 shown on the panel can be pressed to start/stop the motor-lever assembly 2100. The machine status 3012 indicates the operational status of the motor-lever assembly 2100. There are four types of machine status that may be displayed: IDLE, HOMING, RUNNING, or STOPPING. When the START/STOP button 3011 displays the “START” button, the motor-lever assembly 2100 is not started, and the machine status 3012 display “IDLE” status. When the “START” button is pressed, the machine status 3012 will changed to “HOMING” for about 1 sec to indicate the device is checking with the limit sensor 2118 that the driving shaft 2111 together with the drive gear 2103 is at its original start position. The motor-lever assembly 2100 starts working right after the “HOMING” status and the machine status 3012 changes to “RUNNING”, while the START/STOP button 3011 changes to display the “STOP” button. When the “STOP” button is pressed, the motor-lever assembly 2100 is paused, and the machine status 3012 changes to “STOPPING” status for about 1 sec before the motor-lever assembly 2100 is fully paused, thereafter the machine status 3012 changes to the “IDLE” status and the “START” button is displayed. System information such as the version information 3013 of the device firmware is also displayed on the touch panel HMI 3011.

As shown in FIG. 6, the touch panel HMI 3010 displays several ventilation parameters of the BVM system 10, including the TV 3014, the BPM 3015 and the I/E ratio 3016. A user can also adjust each of those ventilation parameters in a predetermined range through the touch panel HMI 3010. In an exemplary prototype built with an Ambu® Oval Silicone Resuscitator (SKU:470003100) together with standard PEEP (Positive End-Expiratory Pressure) valve (SKU: A000213000) and ventilator circuit (SKU: 40401), the TV parameter 3014 may be adjusted between 200 to 800 ml with a step of 200 ml; the BPM 3015 may be adjust between 8-30 BPM with a step of 1 BPM; and the I/E ratio 3016 may be adjusted to be 1:1, 1:2 or 1:3. The adjustment may be done by pressing the incremental button 3017 or decremental button 3018 located above and below the display field of each parameter.

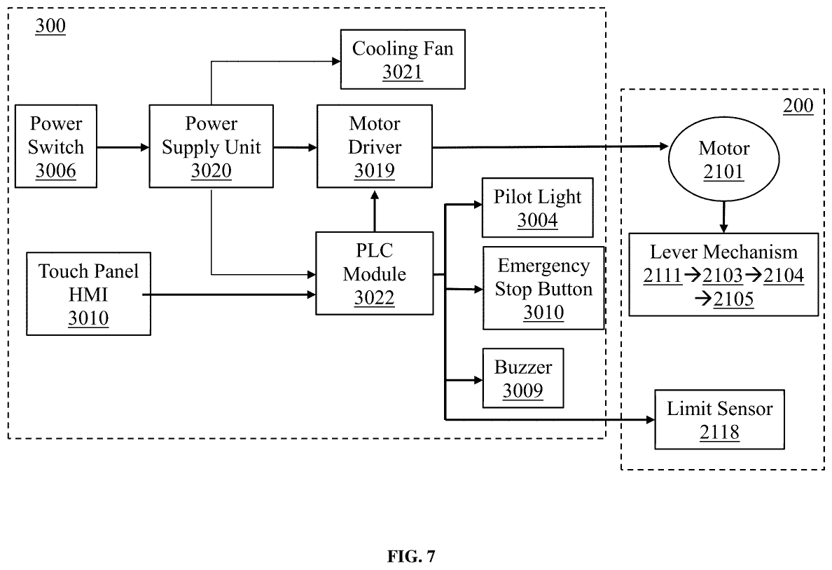

A schematic block diagram of the electronic control unit 300 according to embodiments of the present invention is shown in FIG. 7. A power supply unit 3020 inside the control box supplies power to the cooling fan 3021 located near the heat dissipation window 3005, to the programmable logic controller (PLC) module 3022, and to the motor 2101 via the motor driver 3019 inside the electronic control unit 300. The PLC module 3022 receives user commands and settings of the ventilation parameters from the touch panel HMI 3010 and controls the motor 2101 via the motor driver 3019 to drive the lever mechanism driving shaft 2111, drive gear 2103, the first and second finger levers 2104, 2105, accordingly. The PLC module 3022 also communicates with the limit sensor 2118 to ensure the movement of the drive gear 2103 and hence the finger levers 2104, 2105 does not exceed the predetermined limit, as well as control the safety measures like the pilot light indicator 3007, the buzzer 3008 and the emergency stop button 3009 to make the device respond immediately when there is a device failure or emergency situation.

The present invention has been described herein using specific embodiment for the purpose of illustration only. It will be readily apparent to one of ordinary skill in the relevant art, that the principles of the present invention can be embodied in other ways. Therefore, the present invention should not be regarded as being limited in scope to the specific embodiment disclosed herein, but instead as being fully commensurate in scope as whatever has been claimed.

1. A system for automating the compression and decompression operation of a bag-valve-mask (BVM), comprising:
a compressible BVM;
an automated compression fixture having a receiving structure to seat the compressible BVM, and having a motor-lever assembly for cyclically compressing and decompressing an airbag of the BVM at a repeatable cycle; and
an electronic control unit coupled to the automated compression fixture adapted to receive settings from a user of the BVM to control operation of the automated compression fixture, the electronic control unit supplying power to the automated compression fixture.
